(19) United States
(12) Patent Application Publication
(10) Pub. No.: US 2026/0102127 A1
(43) Pub. Date: Apr. 16, 2026

(54) ADJUSTMENT SYSTEMS AND METHODS FOR COMPUTED TOMOGRAPHY

(71) Applicant: GE Precision Healthcare LLC, Waukesha, WI (US)

(72) Inventors: Arka Datta, Täby (SE); Brandon A. Smith, Waukesha, WI (US); Brian E. Nett, Waukesha, WI (US)

(21) Appl. No.: 18/916,587

(22) Filed: Oct. 15, 2024

(51) Int. Cl. A61B 6/03; A61B 6/04

(52) U.S. Cl. CPC A61B 6/03; A61B 6/0407

(57) ABSTRACT

Methods and systems are provided for automatically adjusting parts of a patient's body relative to a CT scanner to optimize image collection and work flow during a CT scan. In one example approach, a method comprises acquiring image data of a patient on an X-ray imaging system patient support surface; performing an adjustment calculation based on the image data; and adjusting a head holder attached to the patient support surface based on the adjustment calculation, wherein the head holder is adjusted using a motor attached to the head holder.

### TECHNICAL FIELD

The present disclosure relates to computed tomography (CT) devices and methods.

### BACKGROUND

Computed tomography (CT), is a computerized X-ray imaging procedure in which a narrow beam of X-rays is aimed at a patient and rotated around the body, producing signals that are processed a computer to generate cross-sectional images, or “slices.” CT scanners may use a motorized X-ray source that rotates around a circular opening of a donut-shaped structure called a gantry. During a CT scan, a patient lies on a surface that moves through the gantry while the X-ray tube rotates around the patient, sending narrow beams of X-rays through the body. As the X-rays leave the patient, they may be picked up by detectors and transmitted to a computer for processing. Resulting image slices can be displayed individually or stacked together by a computer to generate 3D images of the patient that shows the skeleton, organs, and/or tissues as well as abnormalities or other features for investigation by a physician.

The inventors herein have recognized that sometimes the position and/or orientation of a patient's body relative to the CT scanner may not be optimal, which may lead to imaging artifacts, higher X-ray doses to the patient, and repeated scans, for example. As an example, the inventors herein have recognized that since a patient's eyes may be more sensitive to X-ray radiation from a CT scanner, it may be desirable to be able to adjust the patient relative to the CT scanner and/or adjust the CT gantry or other parts of a CT scanner in order to reduce or minimize X-ray doses delivered to the patient's eyes during a scan.

In some approaches, manual adjustment of a patient undergoing a CT scan and or manual adjustment of aspects of the CT scan may be performed; however, the inventors herein have recognized that manual adjustment may reduce accuracy, not be repeatable, may slow CT scanning workflow and may increase time a patient undergoes scanning, for example.

### SUMMARY

In order to address the above-described and other issues, systems and methods are disclosed herein for automatically adjusting parts of a patient's body (e.g., the patients head) relative to the CT scanner and/or automatically adjusting parts of the CT scanner, e.g., the gantry, relative to the patient's body in order to optimize image collection and work flow during a CT scan. In one example, approach, a method is provided comprising acquiring image data of a patient on an X-ray imaging system patient support surface; performing an adjustment calculation based on the image data; and adjusting a head holder attached to the patient support surface based on the adjustment calculation, wherein the head holder is adjusted using a motor attached to the head holder.

In this way, CT scanning imaging metrics, X-ray dosage and workflow may be improved. For example, X-ray dosage to sensitive areas of the body, such as the eyes, may be reduced or minimized because the position and/or orientation of the patient undergoing the CT scan has been automatically adjusted to an optimal position. Additionally, such an approach may make the CT scanning workflow more efficient by saving time per scan, making the scan results more repeatable, reducing imaging artifacts, and increasing patient throughput.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows an example computed tomography (CT) imaging system, in accordance with the disclosure;

FIG. 2 shows a block schematic diagram of an example CT imaging system, in accordance with the disclosure;

FIG. 3 shows a schematic diagram of an example adjustment calculation system, in accordance with the disclosure;

FIGS. 4A & 4B illustrate example CT scanning adjustment approaches, in accordance with the disclosure;

FIGS. 5 & 6 show example adjustment motor configurations on a head support apparatus, in accordance with the disclosure;

FIGS. 7A & 7B show example 3D imaging camera placements in a CT system, in accordance with the disclosure;

FIG. 8 shows an example method for CT scan patient adjustments, in accordance with the disclosure;

FIG. 9 shows an example method for detecting body part areas of a patient and performing CT scan patient adjustments, in accordance with the disclosure;

FIG. 10 shows an example method for CT scan patient adjustments based on a virtual model, in accordance with the disclosure;

FIG. 11 illustrates a virtual model used to perform CT scan patient adjustments, in accordance with the disclosure;

FIG. 12 illustrates example relationships between a patient support surface tilt angle and an X-ray overlapping area with the patient's eyes used to calculate CT scan patient adjustments, in accordance with the disclosure

### DETAILED DESCRIPTION

Non-invasive imaging technologies, such as CT imaging, allow images of the internal structures or features of a patient to be obtained without performing an invasive procedure on the patient. Such non-invasive imaging technologies rely on various physical principles, such as the differential transmission of X-rays through a target volume or the reflection of acoustic waves, to acquire data and to construct images that may represent internal features of a patient.

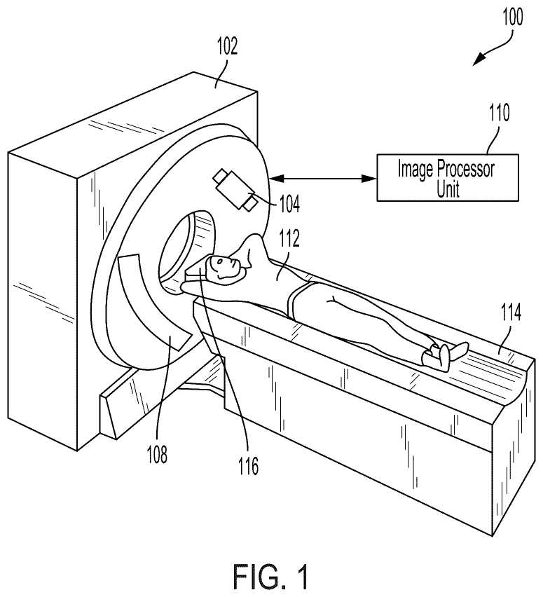

For example, in computed tomography (CT) and other X-ray based imaging technologies, X-ray radiation is used to probe regions of interest, e.g., portions of a human patient. After passing through a region of interest, radiation impacts a detector where the image data is collected. In digital X-ray systems a photodetector may produce signals representative of an amount or intensity of radiation impacting discrete pixel regions of the detector surface. The signals may then be processed to generate an image that may be displayed in a suitable way for review. In the images produced by such systems, it may be possible to identify and examine the internal structures and organs within a patient's body, for example. In CT systems, a detector array, including a series of detector elements, may produce similar signals through various positions as a gantry included in the CT system (see FIG. 1 described below) is displaced around a patient, allowing volumetric reconstructions to be obtained.

As remarked above, the inventors herein have recognized that sometimes the position and/or orientation of a patient's body relative to the CT scanner may not be optimal which may lead to imaging artifacts, higher X-ray doses to the patient, and repeated scans, for example. As an example, the inventors herein have recognized that since a patient's eyes, and/or other sensitive areas, may be more sensitive to X-ray radiation from a CT scanner, it may be desirable to be able to adjust the patient relative to the CT scanner and/or adjust the CT gantry or other parts of a CT scanner in order to minimize X-ray doses delivered to the patient's eyes or other sensitive areas during a scan. Further, in some approaches, manual adjustment of a patient undergoing a CT scan and/or manual adjustment of aspects of the CT scan may be performed; however, the inventors herein have recognized that manual adjustment may reduce accuracy, not be repeatable, may slow CT scanning workflow and may increase time a patient undergoes scanning, for example.

In order to address these and other issues, systems and methods are disclosed herein for automatically adjusting parts of a patient's body (e.g., the patients head) relative to the CT scanner and/or automatically adjusting parts of the CT scanner, e.g., the gantry, relative to the patient's body in order to optimize image collection and work flow during a CT scan.

Turning now to the figures, FIGS. 1-2 and FIGS. 4-8 show example configurations with relative positioning of various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below/underneath one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

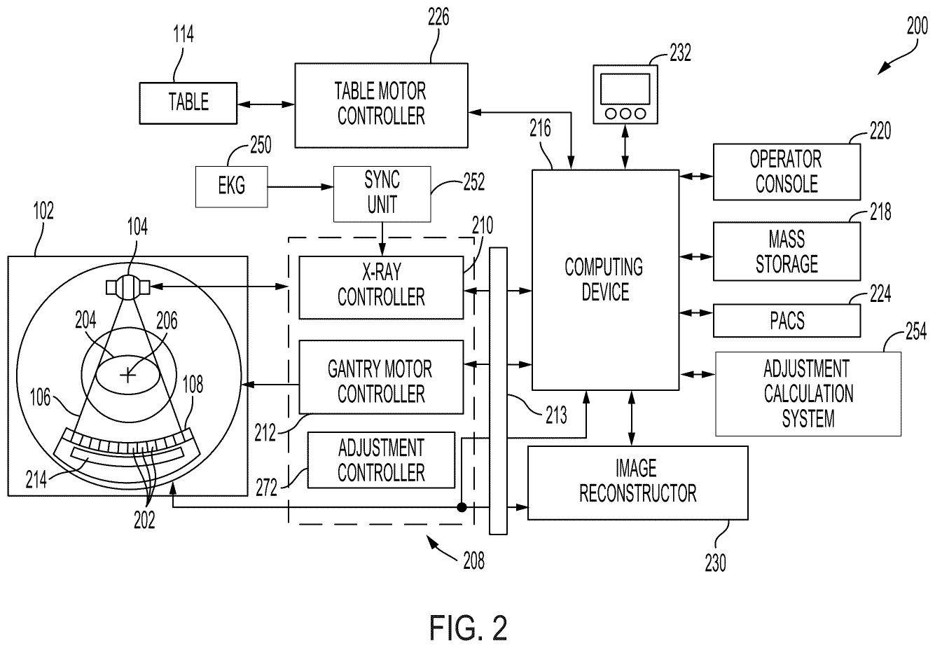

FIG. 1 shows an example CT system 100 configured for CT imaging with photon-counting detectors. CT system 100 may be configured to image a subject 112 such as a patient, an inanimate object, one or more manufactured parts, and/or foreign objects such as dental implants, stents, and/or contrast agents present within the body, for example. The CT system 100 includes a gantry 102, which in turn, may further include at least one X-ray source 104 configured to project a beam of X-ray radiation 106 (see FIG. 2) for use in imaging the subject 112 laying on a table 114. Specifically, the X-ray source 104 is configured to project the X-ray radiation beams 106 towards a detector array 108 positioned on the opposite side of the gantry 102. Although FIG. 1 depicts a single X-ray source 104, in some examples, multiple X-ray sources and detectors may be employed to project a plurality of X-ray radiation beams for acquiring projection data at the same or different energy levels corresponding to the patient. In some embodiments, the X-ray source 104 may enable dual-energy spectral imaging by rapid peak kilovoltage (kVp) switching. In some examples, the X-ray detector employed may be a photon-counting detector which is capable of differentiating X-ray photons of different energies. However, other types of X-ray detectors are contemplated.

In some examples, the CT system 100 further includes an image processor unit 110 configured to reconstruct images of a target volume of the subject 112 using an iterative or analytic image reconstruction method or other suitable reconstruction methods. For example, the image processor unit 110 may use an analytic image reconstruction approach such as filtered back projection (FBP) to reconstruct images of a target volume of the patient. As another example, the image processor unit 110 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the subject 112. In some examples, the image processor unit 110 may use an analytic image reconstruction approach, such as FBP, in addition to an iterative image reconstruction approach.

In some CT imaging system configurations, an X-ray source projects a cone-shaped X-ray radiation beam which is defined with respect to an X-Y-Z Cartesian coordinate system and generally referred to as an “imaging volume.” The X-ray radiation beam passes through an object being imaged, such as the patient or subject. The X-ray radiation beam, after being attenuated by the object, impinges upon an array of detector elements. The intensity of the attenuated X-ray radiation beam received at the detector array is dependent upon the attenuation of an X-ray radiation beam by the object. Each detector element of the array produces a separate electrical signal that is a measurement of the X-ray beam attenuation at the detector location. The attenuation measurements from all the detector elements are acquired separately to produce a transmission profile.

In some CT systems, the X-ray source and the detector array may be rotated with a gantry within the imaging volume and around the object to be imaged such that an angle at which the X-ray beam intersects the object constantly changes. A group of X-ray radiation attenuation measurements, e.g., projection data, from the detector array at one gantry angle may be referred to as a “view.” A “scan” of the object includes a set of views made at different gantry angles, or view angles, during one revolution of the X-ray source and detector.

FIG. 2 illustrates an example imaging system 200 similar to the CT system 100 of FIG. 1. In accordance with aspects of the present disclosure, the imaging system 200 is configured for imaging a subject 204 (e.g., the subject 112 of FIG. 1). In one embodiment, the imaging system 200 includes the detector array 108 (see FIG. 1). The detector array 108 further includes a plurality of detector elements 202 that together sense the X-ray radiation beam 106 that passes through the subject 204 (such as a patient) to acquire corresponding projection data. In some embodiments, the detector array 108 may be fabricated in a multi-slice configuration including the plurality of rows of cells or detector elements 202, where one or more additional rows of the detector elements 202 are arranged in a parallel configuration for acquiring the projection data.

In some examples, the imaging system 200 may be configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along a segment of a circle.

As the X-ray source 104 and the detector array 108 rotate, the detector array 108 collects data of the attenuated X-ray beams. The data collected by the detector array 108 undergoes pre-processing and calibration to condition the data to represent the line integrals of the attenuation coefficients of the scanned subject 204. The processed data are commonly called projections. In some examples, the individual detectors or detector elements 202 of the detector array 108 may include photon-counting detectors which register the interactions of individual photons into one or more energy bins.

The acquired sets of projection data may be used for basis material decomposition (BMD). During BMD, the measured projections are converted to a set of material-density projections. The material-density projections may be reconstructed to form a set of material-density maps or images of each respective basis material, such as bone, soft tissue, and/or contrast agent maps. The density maps or images may be, in turn, associated to form a 3D volumetric image of the basis material, for example, bone, soft tissue, and/or contrast agent, in the imaged volume.

Once reconstructed, the basis material image produced by the imaging system 200 reveals internal features of the subject 204, expressed in the densities of two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discernable in the image based upon the skill and knowledge of the individual practitioner.

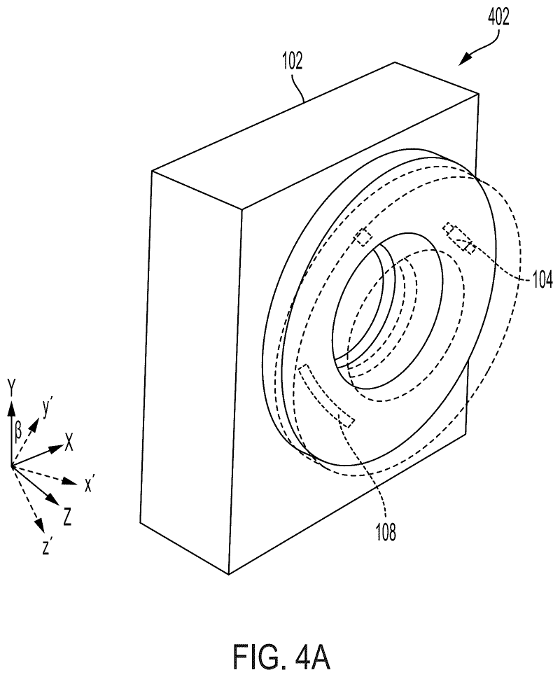

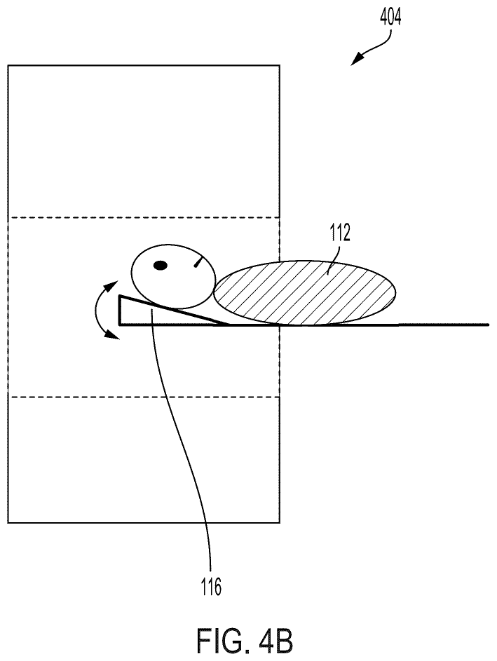

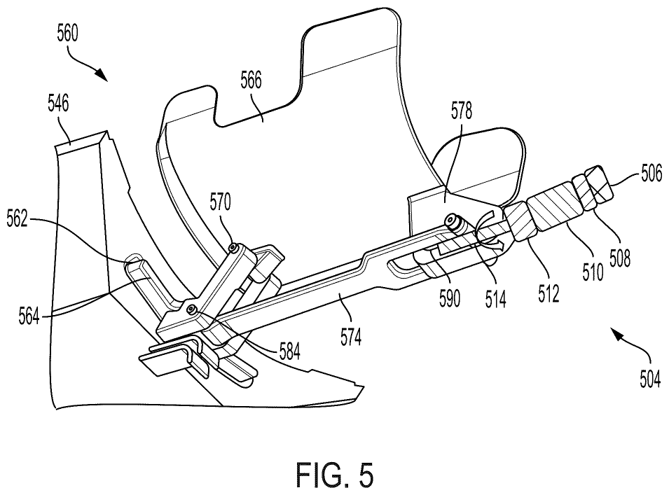

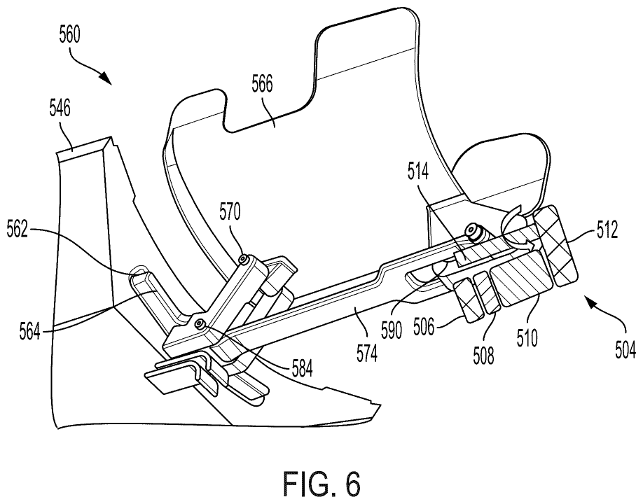

In some examples, the imaging system 200 may include a control mechanism 208 to control movement of the components such as rotation of the gantry 102 and the operation of the X-ray source 104. In some examples, the control mechanism 208 may further include an X-ray controller 210 configured to provide power and timing signals to the X-ray source 104. Additionally, the control mechanism 208 may include a gantry motor controller 212 configured to control a rotational speed and/or position of the gantry 102 based on imaging requirements. In some examples, gantry motor controller 212 may be configured to control a tilt or angle of the gantry in response to target specifications, as shown and described in FIGS. 4A & 4B below. As described in more detail below, control mechanism 208 may additionally include an adjustment controller 272 configured to adjust positions of one or more portions of a patient support surface, such as a patient head holder 116 (shown in FIG. 1). Adjustment controller 272 may be in communication with one or more motors coupled to an adjustable component of the patient surface. For example, as shown in FIGS. 5 and 6 described below, controller 272 may communicate with one or more motors configured to control an angle or tilt and/or rotation or orientation of a head holder 116 in the CT system.

In some examples, the control mechanism 208 my further include a data acquisition system (DAS) 214 configured to sample analog data received from the detector elements 202 and convert the analog data to digital signals for subsequent processing. The DAS 214 may be further configured to selectively aggregate data from a subset of the detector elements 202 into so-called macro-detectors. The data sampled and digitized by the DAS 214 may be transmitted to a computer or computing device 216 via a slip ring 213. In one example, the computing device 216 stores the data in a storage device or mass storage 218. The storage device 218, for example, may be any type of non-transitory memory and may include a hard disk drive, a floppy disk drive, a compact disk-read/write (CD-R/W) drive, a Digital Versatile Disc (DVD) drive, a flash drive, and/or a solid-state storage drive.

Additionally, the computing device 216 provides commands and parameters to one or more of the DAS 214, the X-ray controller 210, and the gantry motor controller 212 for controlling system operations such as data acquisition and/or processing. In some examples, the computing device 216 controls system operations based on operator input. The computing device 216 receives the operator input, for example, including commands and/or scanning parameters via an operator console 220 operatively coupled to the computing device 216. The operator console 220 may include a keyboard (not shown) or a touchscreen to allow the operator to specify the commands and/or scanning parameters.

Although FIG. 2 illustrates one operator console 220, more than one operator console may be coupled to the imaging system 200, for example, for inputting or outputting system parameters, requesting examinations, plotting data, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc.

In some examples, the imaging system 200 either includes, or is coupled to, a picture archiving and communications system (PACS) 224. In an example implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a table 114 which may be a motorized table. Specifically, the table motor controller 226 may move the table 114 for appropriately positioning the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized X-ray data to perform high-speed reconstruction. Although FIG. 2 illustrates the image reconstructor 230 as a separate entity, in some embodiments, the image reconstructor 230 may form part of the computing device 216. Alternatively, the image reconstructor 230 may be absent from the imaging system 200 and instead the computing device 216 may perform one or more functions of the image reconstructor 230. Moreover, the image reconstructor 230 may be located locally or remotely, and may be operatively connected to the imaging system 200 using a wired or wireless network. For example, some embodiments may use computing resources in a “cloud”network cluster for the image reconstructor 230.

In some examples, the image reconstructor 230 stores the images reconstructed in the storage device 218. Alternatively, the image reconstructor 230 may transmit the reconstructed images to the computing device 216 for generating useful patient information for diagnosis and evaluation. In some embodiments, the computing device 216 may transmit the reconstructed images and/or the patient information to a display or display device 232 communicatively coupled to the computing device 216 and/or the image reconstructor 230. In some embodiments, the reconstructed images may be transmitted from the computing device 216 or the image reconstructor 230 to the storage device 218 for short-term or long-term storage.

Information may be transmitted between the components residing in the gantry 102 and external devices (such as the computing device 216 and/or image reconstructor 230) via the slip ring 213, which facilitates electronic communication across the rotating gantry.

Imaging system 200 may further include an adjustment calculation system 254. Adjustment calculation system 254 may be configured to calculate an amount of adjustment to perform (e.g., adjustment of a head support, gantry, or other component of the CT system) in order to automatically adjust parts of a patient's body (e.g., the patients head) relative to the CT scanner and/or automatically adjust parts of the CT scanner, e.g., the gantry, relative to the patient's body in order to optimize image collection and work flow during a CT scan. Various inputs, such as 3D camera data and other inputs may be used by adjustment calculation system 254 to determine an amount and type of adjustment to perform.

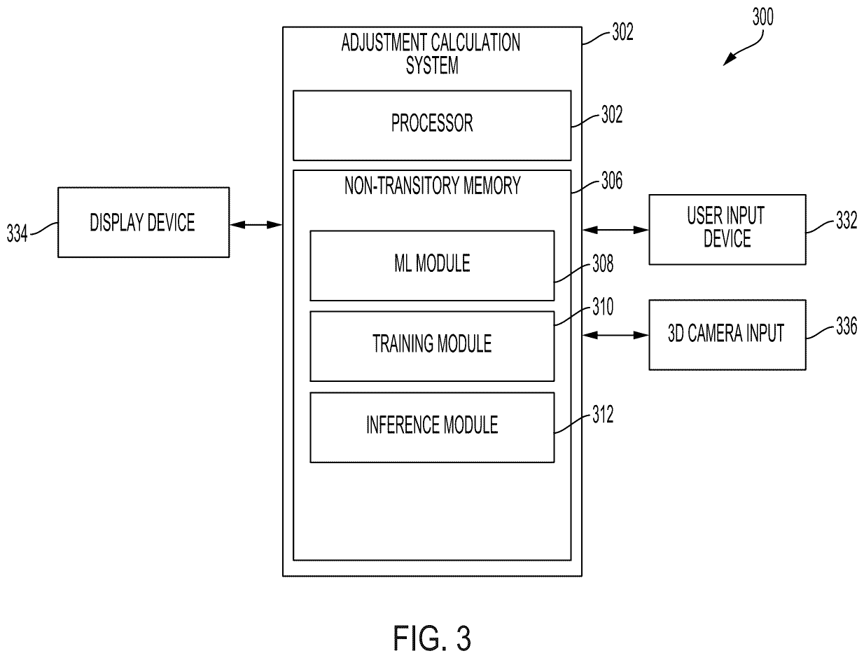

Referring now to FIG. 3, a block diagram 300 shows an adjustment calculation system 302, in accordance with embodiments. In some examples, adjustment calculation system 302 may be incorporated into imaging system 200. In some examples, at least a portion of adjustment calculation system 302 may be disposed at a device (e.g., edge device, server, etc.) communicably coupled to imaging system 200 via wired and/or wireless connections. In some examples, adjustment calculation system 302 may be operably/communicatively coupled to a user input device 332, a display device 334, and/or a 3D camera input 336. For example, user input device 332 may be included within operator console 220 of the imaging system 200, while display device 334 may be included within display device 232 of imaging system 200, at least in some examples. 3D camera input 336 may comprise data output by a camera included in the CT system and used to determine patient position and/or orientation to calculate how much adjustment is to be performed.

Adjustment calculation system 302 includes a processor 304 configured to execute machine readable instructions stored in non-transitory memory 306. Processor 304 may be single core or multi-core, and the programs executed thereon may be configured for parallel or distributed processing. In some examples, processor 304 may optionally include individual components that are distributed throughout two or more devices, which may be remotely located and/or configured for coordinated processing. In some examples, one or more aspects of processor 304 may be virtualized and executed by remotely-accessible networked computing devices configured in a cloud computing configuration.

In some examples, non-transitory memory 306 may store a machine learning (ML) module 308, a training module 310, and an inference module 312, in addition to other functional modules. ML module 308 may include one or more ML models, and instructions for implementing the one or more ML models to calculate an amount of adjustment of a patient support surface or other component to perform. ML module 308 may include models of various types, including trained and/or untrained neural networks such as CNNs, statistical models, or other models, and may further include various data, or metadata pertaining to the one or more models stored therein.

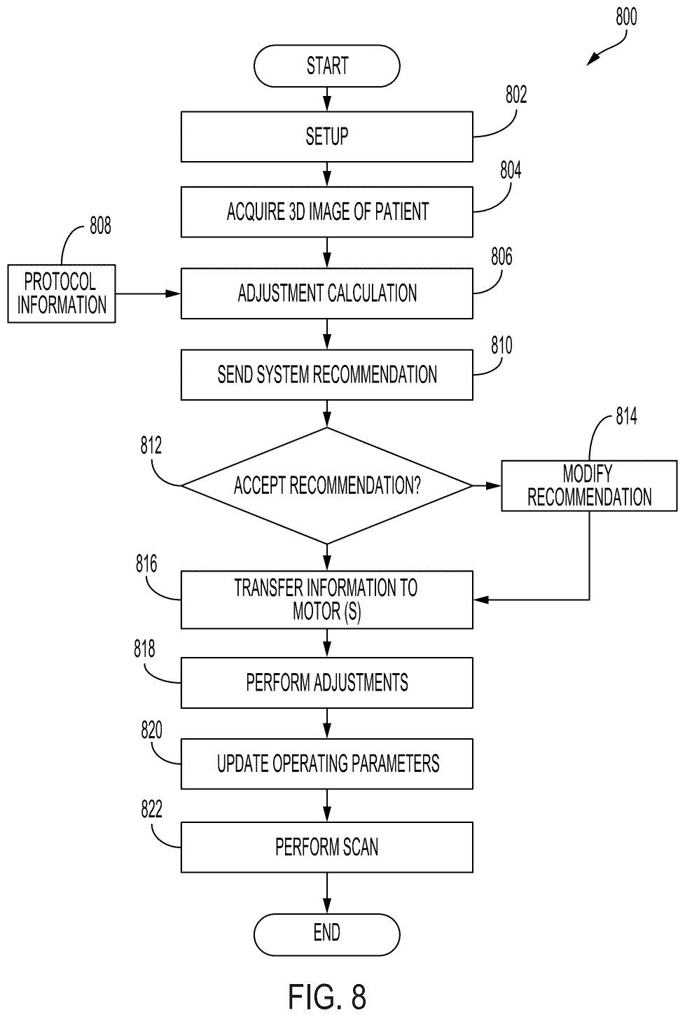

Non-transitory memory 306 may further store a training module 310, which may comprise instructions for training one or more of the models stored in ML module 308. In particular, training module 310 may include instructions that, when executed by processor 304, cause adjustment calculation system 302 to conduct one or more of the steps of methods shown in FIGS. 8-10 described below. In some embodiments, training module 310 may include instructions for implementing one or more gradient descent algorithms, applying one or more loss functions, and/or training routines, for use in adjusting parameters of one or more ML models of ML module 308. Training module 310 may include training datasets for the one or more models of ML module 308.

In some embodiments, non-transitory memory 306 may include components disposed at two or more devices, which may be remotely located and/or configured for coordinated processing. In some embodiments, one or more aspects of non-transitory memory 306 may include remotely-accessible networked storage devices configured in a cloud computing configuration.

User input device 332 may comprise one or more of a touchscreen, a keyboard, a mouse, a trackpad, or other device configured to enable a user to interact with and manipulate data within adjustment calculation system 302. In some examples, user input device 332 may enable a user to modify or implement adjustment recommendations output by the adjustment calculation system 302.

Display device 334 may include one or more display devices utilizing virtually any type of technology. In some embodiments, display device 334 may comprise a computer monitor. Display device 334 may be combined with processor 304, non-transitory memory 306, and/or user input device 332 in a shared enclosure, or may be peripheral display devices and may comprise a monitor, touchscreen, projector, or other display device known in the art.

FIGS. 4A & 4B illustrate example adjustment locations within a CT scanning system that may be used to automatically adjust parts of a patient's body (e.g., the patients head) relative to the CT scanner and/or automatically adjusting parts of the CT scanner, e.g., the gantry, relative to the patient's body in order to optimize image collection and workflow during a CT scan.

At 402 in FIG. 4A, tilting a gantry 102 relative a patient is illustrated. For example, a motor may be coupled with parts of the gantry and controlled by a control system, such as adjustment controller 272 shown in FIG. 2 described above, to change the angle of the gantry and X-ray source and detectors thereon relative to a patient's body or parts of a patient body in order to reduce X-ray dosage to certain areas of the patient.

As another example, at 404, FIG. 4B illustrates tilting a head holder 116 in order to adjust a position, angle, rotation and/or orientation of a patient's head for scanning. Other adjustment locations are contemplated as well. For example, the entire patient table 114 or portions of the patient support surface may be adjusted to change the relative positioning of the patient within the CT imaging system. In some examples, these adjustments may be performed by one or more adjustment motors, such as motor system 504 shown in FIGS. 5 and 6 described below, that control the positioning of the parts to be adjusted based on predetermined calculations, examples of which are described in more detail below.

FIGS. 5 and 6 show example motor systems 504 that may be incorporated into a head support apparatus 560. The motor systems 504 may be used for automated tilting and rotation of the head support prior to head imaging by the CT imaging system. The tilt and rotation angle adjustments may be determined by adjustment calculation system 254 shown in FIG. 2 described above, to improve imaging metrics such as minimizing X-ray dose to at least some areas of the patient and reducing artifacts in the imaging process. As described in more detail below, once determined by the adjustment calculation system 254, the adjustment amount and type (e.g., tilting or rotation) may be communicated automatically to an adjustment controller, such as adjustment controller 272 shown in FIG. 2. The adjustment controller may then cause motor system 504 to automatically adjust the head support apparatus 506 based on the output from the adjustment calculation system 254. The motor system 504 may be positioned outside the imaging area (e.g., the scan field of view, the region of interest) of the CT system and may support the system to tilt and rotate in a continuous range of available angles, for example.

In particular, FIGS. 5 & 6 show example adjustment motor configurations on a head support apparatus 560. As used herein, the terms “head support,” “head support apparatus,” “head rest,” “head support assembly,” and like are used interchangeably and are intended to mean a support surface that is configured to support and stabilize a patient's head in a particular position during a CT imaging or related process. Though examples are shown and described herein for automatically adjusting a head support, it is contemplated that other patient support surfaces may be automatically adjusted in order to position a patient in an optimal location for scanning.

The motor system 504 may be positioned in any suitable location for adjusting tilt, angle, rotation and/or orientation of the head support 560 (or other patient support surface). In the examples described here, the orientation of the motor system is show inline with the head support in FIG. 5 and shown in parallel in FIG. 6.

FIGS. 5 and 6 both show a head support assembly 560 that may be suitable for use in a CT or PET context, for example. By way of a non-limiting example, FIGS. 5 and 6 show the support 560 as having a sliding engagement structure 564 suitable for being inserted into a complementary slot 562 formed at the end of the table 546 so as to form a cantilevered interface when so engaged. In such an embodiment, the engagement structure 564 may be made from a non-metallic, or otherwise X-ray transmissive material (e.g., plastic or carbon fiber), so as to reduce interference with the imaging process (e.g., artifact formation).

In FIGS. 5 and 6, the engagement structure 564, which may remain substantially stationary when in use, may be connected to a tilting head support surface 566 that is configured to pivot (e.g., via non-metallic pivot pin 570) with respect to the engagement structure 564. In FIGS. 5 and 6, the head support is shown tilted at an angle of approximately 30° with respect to the engagement structure 564. The tilting head support surface 566 may be configured to support the head of a patient during imaging and may be automatically and/or manually adjusted before and/or during the scanning process. In some examples, the head support surface 566 may also be made of a non-metallic, X-ray transmissive material (e.g., plastic or carbon fiber) so as to not interfere with imaging of the head and brain, for example. In some examples, straps (e.g., hook and loop straps) may be used to secure one or both of the patient's chin or forehead to the head support surface 566 during imaging.

In some examples, an adjustment arm 574 may provide a linkage between the engagement structure 564 and an adjustment mechanism controlled by motor system 504. In some examples, adjustment of the head support 560 may only be performed by motor system 504 so that no manual control of the adjustment is possible. However, in other examples, an operator may be able to override the motor-driven adjustment by adjusting components of the head support manually. By way of example, the adjustment arm 574 may be pivotally fixed (e.g., such as using a non-metallic pivot pin 584) to the engagement structure 564 and on another end, slidably engaged with the head support surface 566 (such as via a support block 578) so as to transfer the support load from the adjustment mechanism to the engagement structure 564. The position of the adjustment arm 574 relative to the support block 578, and thus to the head support surface 566, may be adjusted by the motor system 504, which may be used to control the extent to which the adjustment arm 574 extends into a slot 590 of the support block 578. The driveshaft 514 also resides within the slot 590 of the support block 578, such that the arm 574 can translate within the slot 590 by rotation of the driveshaft 514. As with other components that may extend into the imaged volume, the adjustment arm 574 may also be made of a non-metallic, X-ray transmissive material (e.g., plastic or carbon fiber) so as to not interfere with imaging process. Further, in some examples, motor system 504 may be positioned outside a range of the X-rays used during a scan, thus may be located at a distal end of the head support above the top of a patient's head, for example.

As illustrated in FIGS. 5 and 6, motor system 504 may comprise a motor 510 coupled to a driveshaft 514. In some examples, a gearbox 512 may be included to translate torque from motor 510 into a target torque amount. In some examples, a brake 508 may be included to reduce back-driving of the motor/driveshaft. In some examples, a rotary encoder may be integrated into the motor assembly for feedback on the actual vs. commanded position. In some examples, a wireless controller 506 may also be included. The wireless controller may be configured to receive signals output by an adjustment calculation system (such as system 302 described above) to implement a target adjustment of tilt, angle, rotation, and/or orientation of the patient support surface.

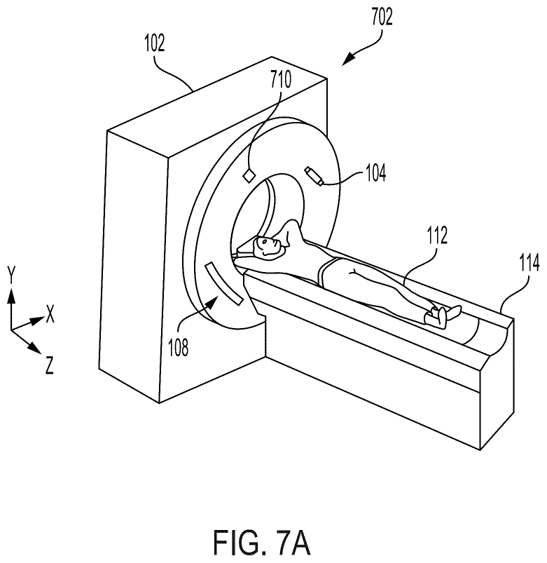

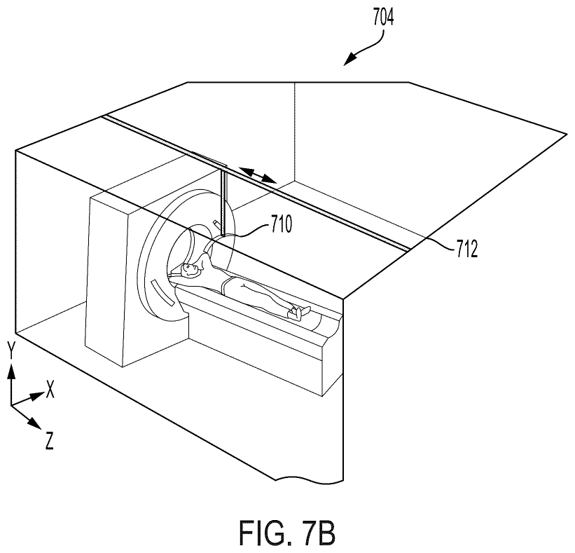

In order to calculate an amount and type of adjustment to perform, e.g., an amount and type of adjustment to be made to a head holder in a CT system, an imaging instrument may be used to determine a current position and orientation of the patient. Such an imaging instrument may be located in any suitable location to capture a patient's position within the CT scanning system. FIGS. 7A & 7B show nonlimiting example imaging instrument placements that are contemplated. At 702, FIG. 7A shows imaging instrument 710 positioned on a gantry 102 above a patient 112 on the CT system table 114. As another example, FIG. 7B shows at 704, an imaging instrument 710 positioned on an instrument guide rail system 712. In this example, the imaging instrument 710 may be positioned at different locations above a patient in order to map the position and orientation of the patient to calculate the adjustments to be made. For example, at 704 imaging instrument 710 may be slideably coupled to rail 712 and may have its position along the rail be manually or automatically adjusted to capture the location and orientation of the patient.

The imaging instrument 710 may comprise any suitable instrument used to capture location and/or orientation of the patient. For example, imaging instrument 710 may comprise a 3D camera such as a laser triangulation/3D profiling camera, a time-of-flight camera, a stereo vision camera, or a structured light camera. This external 3D camera system may be used to map the patient head (or other body part region) and calculate the head (or body part) orientation (x, y, z, Theta, Phi, Gamma) and anatomical feature locations, for example using segmentation as described below. The differential from the desired position may then be calculated and an appropriate signal may then be transferred to the motor system 504 to adjust the head support for optimal imaging, dosing and workflow.

FIG. 8 shows an example method for CT scan patient adjustments that may be used for automatically adjusting parts of a patient's body (e.g., the patients head) relative to the CT scanner and/or automatically adjusting parts of the CT scanner, e.g., the gantry, relative to the patient's body in order to optimize image collection and workflow during a CT scan.

At 802, method 800 includes performing setup procedures. For example, step 802 may include positioning a patient on the CT scan table (e.g., table 114 shown in FIG. 1) and head holder (e.g., head holder 116), performing scout scans, landmarking various parts of the patient to identify regions for scanning, entering protocol information (e.g., focal spot, etc.) into the scanning software, etc.

At 804, method 800 includes acquiring a 3D image of the patient. For example imaging instrument 710 shown in FIG. 7A or 7B may be used to capture 3D data of the patient to determine position and/or orientation to map the patient head (or other body part region) and calculate the head (or body part) orientation (x, y, z, Theta, Phi, Gamma). This data may be used to perform body part identification and to adjust/optimize the angle of the gantry and/or the angle or rotation of the head holder to reduce the dose received to a patient's eyes or other sensitive areas, such as breast tissue, for example.

At 806, method 800 includes performing an adjustment calculation. In some examples, the adjustment calculation may comprise a tilt/rotation calculation for the gantry or head holder or other patient support surface. The adjustment calculation may be performed by adjustment calculation system 302 described above. The adjustment calculation may be based on the 3D image data of the patient and other parameters, including calculations that determine or identify sensitive regions of the patient, such as the eyes. Examples of parameters that may be used in the adjustment calculation are described in more detail below.

In some examples, adjustment of the patient support surface (e.g., a head holder) or gantry to optimize position of the patient for scanning may be performed automatically based on the adjustment calculation. For example, motor system 504 may be automatically initiated by a computing device in the system to make the adjustment determined by the adjustment calculation. However, in some examples, the system may send an adjustment recommendation to an operator of the CT scanning system so that the operator can accept or modify the target adjustment. Thus at 810, method 800 may include sending system recommendations indicating an amount of adjustment recommended to be performed to optimize patient position or orientation for a CT scan. These recommendations may be displayed on a display device or output in some other suitable way, e.g., via an audio or haptic output indicating an amount of target adjustment, for example.

At 812, method 800 may include determining if the adjustment recommendation is accepted. For example, an operator may review the adjustment recommendation output by the adjustment calculation system and determine if the recommendation seem appropriate. If recommendation is not accepted at 812, method 800 proceeds to 814 to modify the adjustment recommendation. For example, the operator may input modifications to the adjustment recommendations or the operator may adjust parameters input in the adjustment calculation system to recalculate the recommended adjustment until the operator is satisfied with the output. In some examples, the operator may perform these inputs via a user interface displayed on a display device.

If the recommendation is accepted at 812 or modified at 814 then method 800 proceeds to 816 to transfer adjustment information to one or more motors in the system to adjust positions, e.g., angle and/or rotation, of the head holder or gantry or other patient support surface. For example, adjustment calculation system 254 may output operating instructions to adjustment controller 272 to cause motor system 504 to adjust the patient support surface. In some examples, the adjustment information may be transferred via wired or wireless methods (e.g., via Wi-Fi, Bluetooth, or other radio wave-based communication method).

At 818, method 800 includes performing the adjustments via one or more motors to adjust the angle and/or rotation of a patient support surface, e.g., a head holder, or an angle and/or rotation of a gantry in the system. At 820, method 800 includes updating operating parameters based on the amount and type of adjustment performed. For example, tube current (mA) tables and/or other imaging parameters may be updated based on the updated position of the patient support surface or gantry in the system. The X-ray tube current (mA) may determine the rate at which X-rays are produced in the X-ray tube (i.e., photons per second). The total number of X-ray photons that are acquired in the 1000 or so projections as the X-ray tube rotates 360° degrees is proportional to the product of the mA and the rotation time (seconds), or the mAs. Since the X-ray tube rotation is normally fixed, the number of photons used to make any CT image is directly proportional to the tube current (mA).

At 822, method 800 includes performing a scan, e.g., a CT imaging scan. In some examples, data from the scan may be reconstructed or reformatted based on the amount and type of adjustments made.

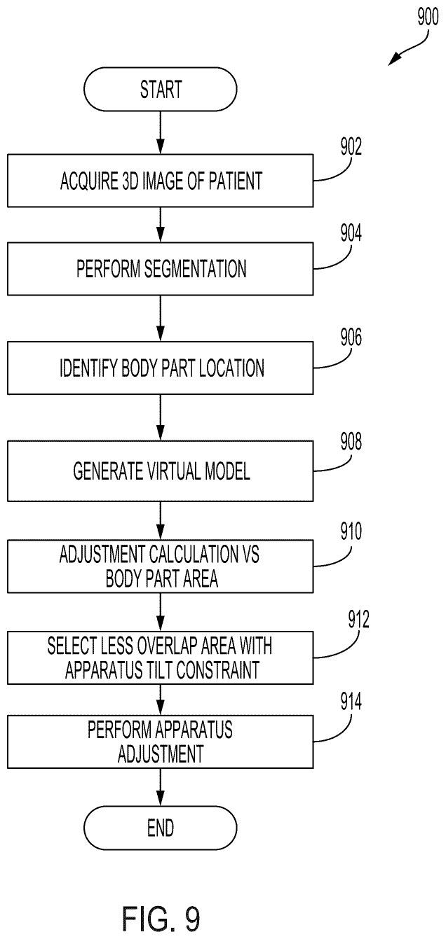

FIG. 9 shows an example method 900 for detecting body part areas of a patient and performing CT apparatus adjustment in response to that detection. The detection of the body part may be used to identify sensitive regions, such as the eyes, so that adjustments can be made to the patient's position to minimize X-ray dose to the sensitive regions.

As remarked above, eyes are more sensitive to X-ray radiation and sometimes the CT gantry may be tilted to minimize the dose delivered to the eyes. This method has typically been manual with accuracy depending on the operator. This disclosure provides automated methods to realize X-ray dose minimization effectively. These methods use external image data (using a 3D camera, for example) to identify head orientation and tilt and/or rotate the gantry or head holder or other patient support surface (with respect to x-y plane) to minimize eye exposure to X-rays during a scan. When tilting, the reconstruction may still be reformatted appropriately for similar orientation for radiologists, for example. The methods can be further generalized for other dose sensitive organs such as breast tissue.

At 902, method 900 include acquiring image of patient. The image data may be any suitable image data that can be used for identifying patient body parts in the data. For example, the image data may comprise 3D image data, images from a plurality of cameras, images from a single camera, scout image data, etc. For example imaging instrument 710 shown in FIG. 7A or 7B may be used to capture 3D data of the patient to determine position and/or orientation to map the patient head (or other body part region) and calculate the head (or body part) orientation (x, y, z, Theta, Phi, Gamma). This data may be used to perform body part identification and to adjust/optimize the angle of the gantry and/or the angle or rotation of the head holder to reduce the dose received to a patient's eyes or other sensitive areas, such as breast tissue, for example.

At 904, method 900 includes performing segmentation based on the 3D image data of the patient in order to identify specific regions of the patient's body that may be more sensitive to X-rays, such as the eyes, breasts, etc. Identifying a body part location within the 3D image data may be performed in a variety of ways using any suitable method.

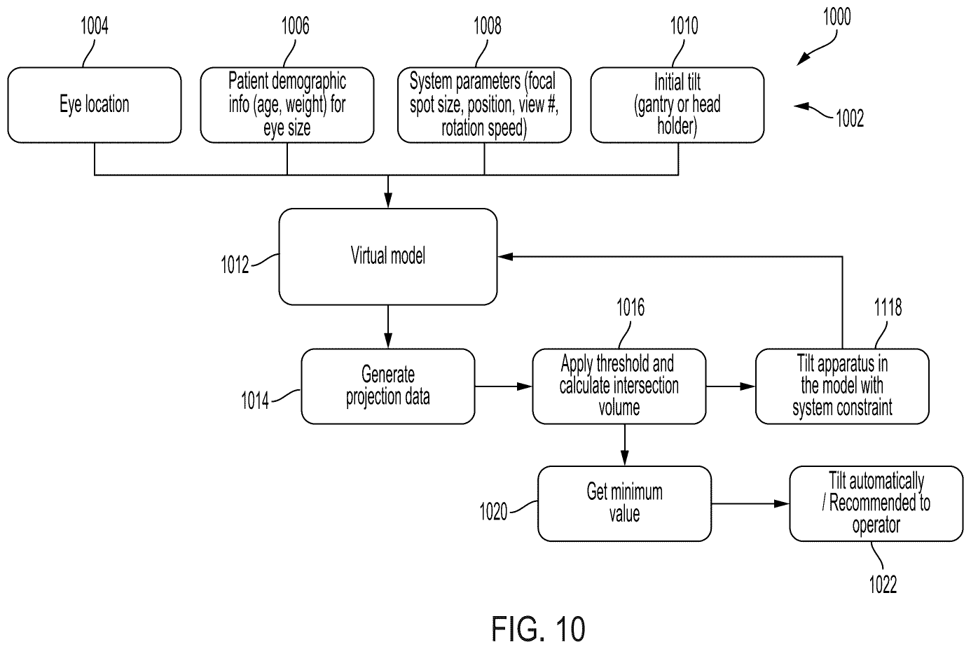

At 906, method 900 includes identifying body part location(s) based on the segmentation process and then at 908, method 900 includes generating a virtual model based on the output of the segmentation process and other parameters. The virtual model may comprise a simulation of at least a portion of a patient's body relative to aspects of the CT scanner, which may be used to calculate an amount of adjustment of the patient to perform. The virtual model is described in more detail below with reference to FIG. 10.

At 910, method 900 includes performing a tilt/rotation calculation vs. body part area (vs. critical organ overlap area). This calculation may be used to find an optimal adjustment amount for a patient support surface within movement thresholds of the apparatus. Thus at 912, method 900 includes selecting less overlap area with an apparatus tilt constraint, and at 914, method 900 includes performing apparatus adjustment to position the patient optimally within the CT imaging system.

FIG. 10 shows an example method 1000 for adjusting patient position within a CT system based on a virtual model or simulation of a patient relative to aspects of the CT scanner or X-ray imaging region. At 1002, method 1000 includes inputting various parameters into a virtual model. For example, at 1004 method 1000 may include inputting eye location, or other body part location identified during a segmentation process, for example, into the virtual model. At 1006, method 1000 includes inputting patient demographic information such as age, weight etc. These parameters may be used to estimate or calculate eye or other body part size to include in the virtual model for a simulation. At 1008, method 1000 includes inputting system parameters into the virtual model. Examples of system parameters that may be input include focal spot size, position, view number, rotation speed, etc. At 1010, method 1000 includes inputting initial tilt (of the gantry or head holder or other patient support surface) into the virtual model. These parameters may provide a baseline for the adjustment calculation system to determine what type (e.g., tilt and/or rotation) and how much of an adjustment of the patient support surface to perform to optimize operation of the CT scanner.

At 1012, method 1000 includes generating a virtual model or simulation based on the parameter inputs. At 1014, method 1000 includes generating projection data to determine where and how X-rays may intersect parts of the patient's body during a scan. At 1016, method 1000 includes applying a threshold and calculating intersection volume. The threshold may be applied to isolate the signal from noise in projection data, for example. At 1018, method 1000 includes tilting and/or rotating the apparatus in the model within system constrains and returning back to adjusting the virtual model at 1012. At 1020, method 1000 includes getting a minimum value from the simulation or virtual model process, i.e., finding the amount and type of adjustment that would minimize X-ray exposure to parts of the patient during a scan. At 1022, method 1000 includes automatically tilting and/or rotating the patient support surface (or gantry) via a motor or outputting an adjustment recommendation to an operator.

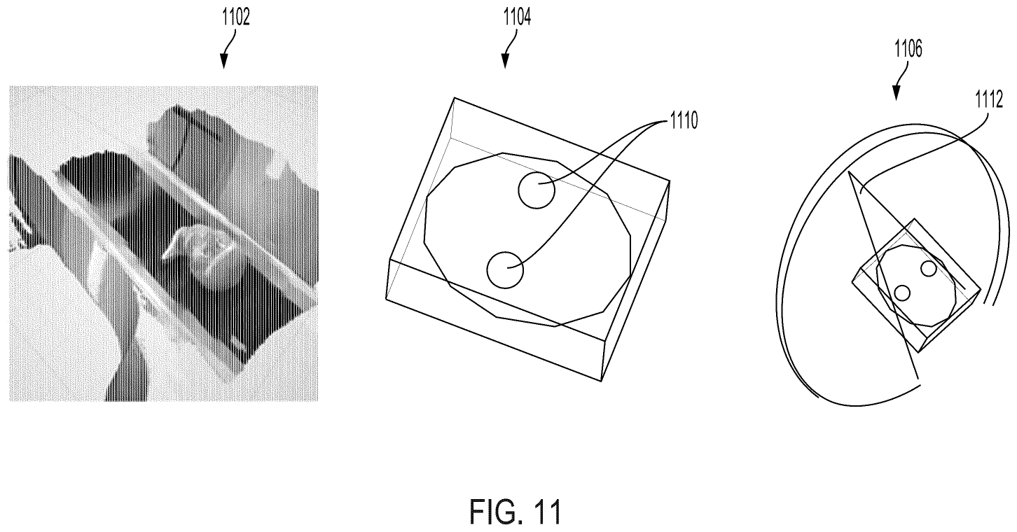

FIG. 11 illustrates aspects of the methods described above and shows at 1102 a 3D image of a head phantom that may be obtained by a 3D camera, for example. At 1104, an input model representing eye regions 1110 is shown. For example, the identified eye regions may be obtained from a segmentation process applied to the 3D image data as described above. This data may be input in a virtual model or simulation, an example of which is shown at 1206, that is used to simulate and calculate an amount of adjustment to perform on a patient support surface (or gantry).

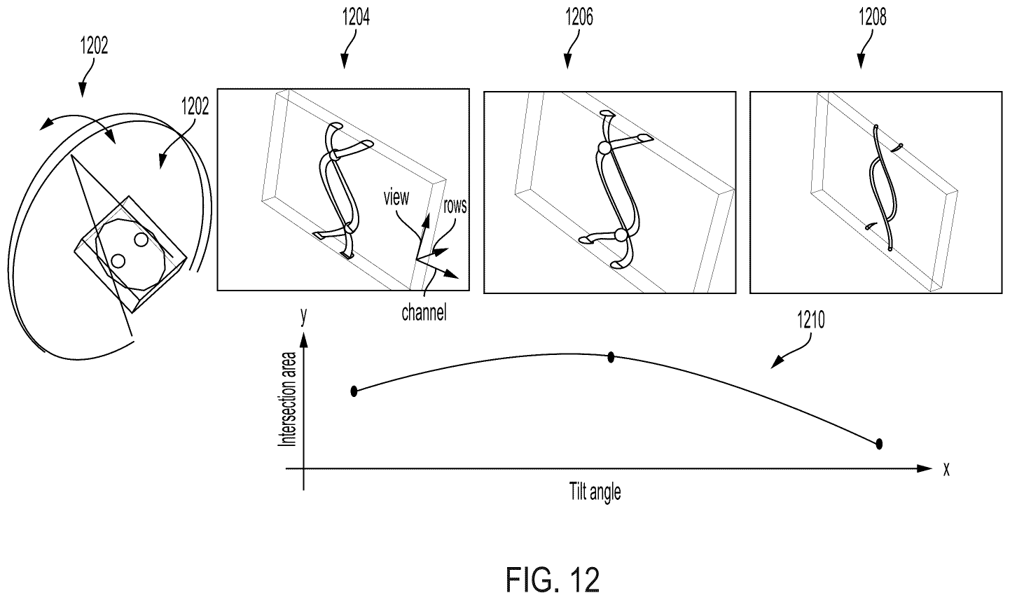

FIG. 12 illustrates aspects of the methods described above and shows relationships between a tilt angle (of a head holder, for example) and X-ray overlapping area with a patient's eyes. At 1202, FIG. 12 shows a simulation model of tilt angle calculated by the adjustment calculation system based on 3D image data of the patient. At 1204, 1206, and 1208, FIG. 12 shows example 3D sonograms generated at different tilt angles. At 1210, FIG. 12 shows a graph of intersection area (on vertical y-axis) plotted against tilt angle (on horizontal x axis). This graph illustrates the relationship between tilting angle and X-ray overlapping area with eyes, for example. The graph corresponds to the three-tilt angle shown in 1204, 1206, and 1208. The range of the tilt angle used was −30 degrees, 0 degrees, and 30 degrees (respectively), where 0 degrees corresponds to no tilt.

The invention will be further described in the following paragraphs. In one aspect, a method for automatically adjusting patient position in an X-ray imaging system is provided that comprises acquiring 3D image data of a patient on an X-ray imaging system patient support surface; performing an adjustment calculation based on the 3D image data; and adjusting at least a portion of the patient support surface based on the adjustment calculation.

In some examples, the X-ray imaging system may comprise a computed tomography (CT) system. The patient support surface may comprise a head holder. In some embodiments, the method may further comprise identifying locations of a patient body part in the 3D image data and performing the adjustment calculation based on the identified locations. In some examples, the body part may comprise the patient's eyes. In some examples, identifying locations of a patient body part in the 3D image data may comprise performing a segmentation process on the 3D image data. In some examples, adjusting at least a portion of the patient support surface may comprise tilting and/or rotating the patient support surface with a motor coupled to the patient support surface. In some examples, adjusting at least a portion of the patient support surface may comprise tilting and/or rotating a gantry of the X-ray imaging system. In some embodiments, the method may further comprise generating a virtual model of the patient relative to the X-ray imaging system based on the 3D image data, wherein performing the adjustment calculation may comprise calculating a differential in the virtual model from a target patient position. In some examples, the method may further comprise updating the X-ray imaging system operating parameters based on the adjustment of the patient support surface. In some examples, updating the X-ray imaging system operating parameters may comprise performing an mA table modification. In some examples, the method may further comprise outputting an adjustment recommendation to a display device based on the adjustment calculation. In some examples, the method may further comprise wirelessly outputting adjustment instructions to a motor coupled to the patient support surface based on the adjustment calculation.

In another aspect, a computed tomography (CT) imaging system is provided that comprises a 3D imaging system configured to capture 3D image data of a patient on a CT imaging system patient support surface; an adjustment calculation system configured to perform an adjustment calculation based on the 3D image data; and a motor coupled to the patient support surface configured to receive adjustment instructions from the adjustment calculation system. In some examples, the patient support surface may comprise a patient head holder. In some examples, the motor may be coupled to the patient support surface at a location outside of a CT imaging area. In some examples, the motor may be configured to automatically adjust an angle and/or rotation of the patient support surface in response to instructions received from the adjustment calculation system.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative and should not be construed to be limiting in any manner.

1. A method for automatically adjusting patient position in an X-ray imaging system, comprising:
acquiring image data of a patient on an X-ray imaging system patient support surface;
performing an adjustment calculation based on the image data; and
adjusting a head holder attached to the patient support surface based on the adjustment calculation, wherein the head holder is adjusted using a motor attached to the head holder.

2. The method of claim 1, wherein the X-ray imaging system comprises a computed tomography (CT) system.

3. The method of claim 1, wherein the image data comprises 3D image data.

4. The method of claim 1, further comprising identifying locations of a patient body part in the image data and performing the adjustment calculation based on the identified locations.

5. The method of claim 4, wherein the body part comprises the patient's eyes.

6. The method of claim 4, wherein identifying locations of a patient body part in the image data comprises performing a segmentation process on the image data.

7. The method of claim 1, wherein adjusting the head holder comprises tilting and/or rotating the head holder using the motor coupled to the head holder.

8. The method of claim 1, further comprising tilting a gantry of the X-ray imaging system.

9. The method of claim 1, further comprising generating a virtual model of the patient relative to the X-ray imaging system based on the image data and wherein performing the adjustment calculation comprises calculating a differential in the virtual model from a target patient position.

10. The method of claim 1, further comprising updating the X-ray imaging system operating parameters based on the adjustment of the patient support surface.

11. The method of claim 10, wherein updating the X-ray imaging system operating parameters comprises performing an mA table modification.

12. The method of claim 1, further comprising outputting an adjustment recommendation to a display device based on the adjustment calculation.

13. The method of claim 1, further comprising outputting adjustment instructions to the motor coupled to head holder based on the adjustment calculation.

14. A method for automatically adjusting patient position in a computed tomography (CT) system, comprising:
acquiring image data of a patient on an X-ray imaging system patient support surface;
performing a segmentation process on the image data to identify locations of a patient body part in the image data;
performing an adjustment calculation based on the image data and the identified patient body part locations; and
adjusting a head holder coupled to the patient support surface based on the adjustment calculation, wherein the head holder is adjusted using a motor coupled to the head holder.

15. The method of claim 14, wherein the image data comprises 3D image data.

16. The method of claim 14, wherein adjusting the head holder comprises tilting and/or rotating the head holder using the motor coupled to the head holder.

17. A computed tomography (CT) imaging system, comprising:
an imaging system configured to capture image data of a patient on a CT imaging system patient support surface;
an adjustment calculation system configured to perform an adjustment calculation based on the image data; and
a motor coupled to a head holder coupled to the patient support surface configured to receive adjustment instructions from the adjustment calculation system.

18. The system of claim 17, wherein the imaging system comprises a 3D imaging system and the image data comprises 3D image data.

19. The system of claim 17, wherein the motor is coupled to head holder at a location outside of a CT imaging area.

20. The system of claim 17, wherein the motor is configured to automatically adjust an angle and/or rotation of the head holder in response to instructions received from the adjustment calculation system.
